(19) United States
(12) Patent Application Publication
(10) Pub. No.: US 2007/0265564 A1
(43) Pub. Date: Nov. 15, 2007

(54) Catheter Having Non-Blood-Contacting Exit Markers

(75) Inventors: Aoifa Mary Daly, Galway (IE); Luke Hughes, Galway (IE); Marcia Gallagher, Galway (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/383,249

(22) Filed: May 15, 2006

(51) Int. Cl. A61M 29/00; A61M 31/00; A61M 37/00

(52) U.S. Cl. 604/103.1

(57) ABSTRACT

A catheter includes elongate flexible inner and outer tubular lumens having proximal and distal regions, and a plurality of exit markers formed in the proximal region. The inner tubular lumen includes an outer surface, and an inner surface, the inner surface defining a guidewire passageway. The outer tubular lumen includes an elongate, flexible, and substantially transparent outer tubular lumen having an outer surface, and an inner surface surrounding the inner tubular lumen. The exit markers may be formed on the inner tubular lumen outer surface or on the outer tubular lumen inner surface, the exit markers being visible to the naked eye through the substantially transparent outer tubular lumen outer surface. The exit markers may also be integrally formed from a polymer that forms a portion of the proximal region of the outer tubular lumen or the inner tubular lumen.

### TECHNICAL FIELD

The present invention generally relates to intravascular catheters, and more particularly to a catheter that is equipped in the vicinity of the catheter distal end with at least one exit marker for viewing the catheter's location using imaging technology.

### BACKGROUND

In a typical percutaneous transluminal coronary angioplasty (PTCA) procedure, a guiding catheter is percutaneously introduced into the cardiovascular system of a patient. The guide catheter is advanced through a vessel until the distal end thereof is at a desired location in the vasculature. A guide wire and a dilatation catheter having a flexible and expandable balloon on the distal end thereof are introduced into the guiding catheter with the guidewire sliding through the dilatation catheter. The guide wire is first advanced out of the guiding catheter into the patient's coronary vasculature, and the dilatation catheter is then advanced over the previously advanced guide wire until the dilatation balloon is properly positioned across the lesion. Once in position, the preformed balloon is inflated to a predetermined size with a liquid or gas at relatively high pressure (e.g. up to twelve atmospheres) to radially compress the arthrosclerotic plaque in the lesion against the inside of the artery wall and thereby dilate the lumen of the artery. The balloon is then deflated to a small profile so that the dilatation catheter may be withdrawn from the patient's vasculature and blood flow resumed through the dilated artery.

Restenosis may occur in an artery following PTCA or other angioplasty procedure. Restenosis is a re-narrowing of the treated coronary artery that is related to the development of neo-intimal hyperplasia within the artery in response to mechanical intervention within a vascular structure. To prevent restenosis and strengthen the treated vascular area, an intravascular prosthesis generally referred to as a stent may be implanted for maintaining vascular patency inside the artery at the lesion. The stent is mounted in a pre-deployment or compressed state around a deflated balloon, and the balloon/stent assembly is maneuvered through a patient's vasculature to the site of a target lesion. The stent is then expanded to a larger diameter for implantation in the vasculature. The stent effectively overcomes the natural tendency of the vessel walls of some patients to close back down, thereby maintaining a normal flow of blood through the vessel that would not be possible if the stent was not in place.

Typically, the proximal section of a balloon catheter includes a plurality of exit markers that are printed or otherwise located on the outer shaft. The exit markers are located at exact distances from the catheter distal end. While the catheter is advanced through a patient's vasculature toward a lesion, a physician can see the exit markers before they enter the patient and know how much catheter length has been inserted into the patient. For example, the markers allow the physician to know exactly how far it is from the catheter's point of entry to the lesion.

Locating the exit markers on the outer shaft has some advantages and disadvantages. Since the exit markers are in plain view, the physician can readily see the markers before they are advanced into the catheter point of entry. The markers are blood-contacting, however, and consequently must be made from a material that is approved by a regulatory agency as biocompatible. Some approved materials are costly or inefficient to incorporate as an exit marker. Further, obtaining regulatory approval for newly introduced materials for use as exit markers requires a great deal of time and cost.

Accordingly, it is desirable to provide a catheter that includes exit markers in a manner that prevents blood contact with the markers. It is also desirable to provide a manufacturing process in which exit markers are easily located on catheter regions that are non-blood contacting. Furthermore, other desirable features and characteristics of the present invention will become apparent from the subsequent detailed description and the appended claims, taken in conjunction with the accompanying drawings and the foregoing technical field and background.

### BRIEF SUMMARY

According to one aspect of the invention, a catheter having non-blood-contacting exit markers is provided. The catheter includes elongate flexible inner and outer tubular lumens having proximal and distal regions, and a plurality of exit markers formed in the proximal region. The inner tubular lumen includes an outer surface, and an inner surface, the inner surface defining a guidewire passageway. The outer tubular lumen includes an elongate, flexible, and substantially transparent outer tubular lumen having an outer surface, and an inner surface surrounding the inner tubular lumen.

According to one embodiment, the exit markers are formed on the inner tubular lumen outer surface, the exit markers being visible to the naked eye through the substantially transparent outer tubular lumen. According to another embodiment, the exit markers are formed on the outer tubular lumen inner surface, the exit markers being visible to the naked eye through the substantially transparent outer tubular lumen outer surface. According to yet another embodiment, the exit markers are integrally formed from a polymer that forms a portion of the outer tubular lumen or the inner tubular lumen.

### BRIEF DESCRIPTION OF THE DRAWINGS

The present invention will hereinafter be described in conjunction with the following drawing figures, wherein like numerals denote like elements, and

FIG. 1 is a side view depicting a balloon catheter assembly coupled to a hub having inflation and guidewire ports, the balloon catheter assembly being illustrated as a cross-sectional view;

FIG. 2 is a cross-sectional view of an over-the-wire catheter;

FIG. 3 is a cutaway perspective view depicting an over-the-wire catheter having exit markers on an inner tubular lumen exterior wall according to an embodiment of the invention;

FIG. 4 is a cutaway perspective view depicting an over-the-wire catheter having exit markers on an outer tubular lumen interior wall according to an embodiment of the invention;

FIG. 5 is a cross-sectional view depicting an over-the-wire catheter including dual-layered inner and outer tubular lumens;

FIG. 6 is a cross-sectional longitudinal view depicting a dual-layered outer tubular lumen for an over-the-wire catheter, the lumen having exit markers formed on an outer lumen inner layer according to an embodiment of the invention; and

FIG. 7 is a cross-sectional longitudinal view depicting an over-the-wire catheter including dual-layered inner and outer tubular lumens, the inner lumen having exit markers formed on an inner lumen outer layer according to an embodiment of the invention.

### DETAILED DESCRIPTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

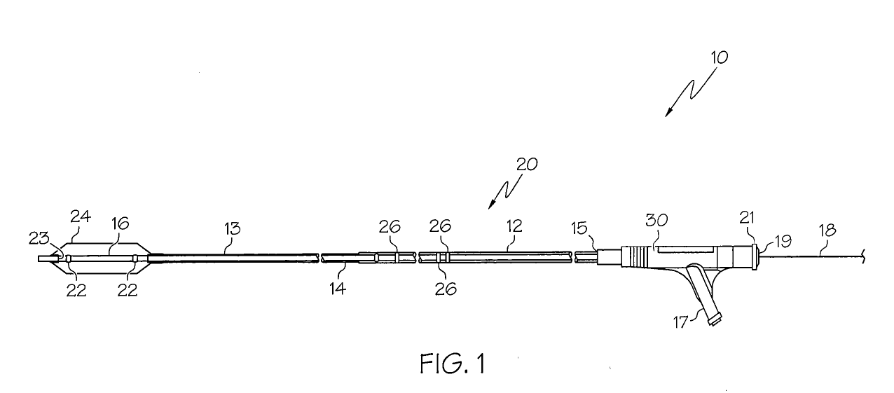

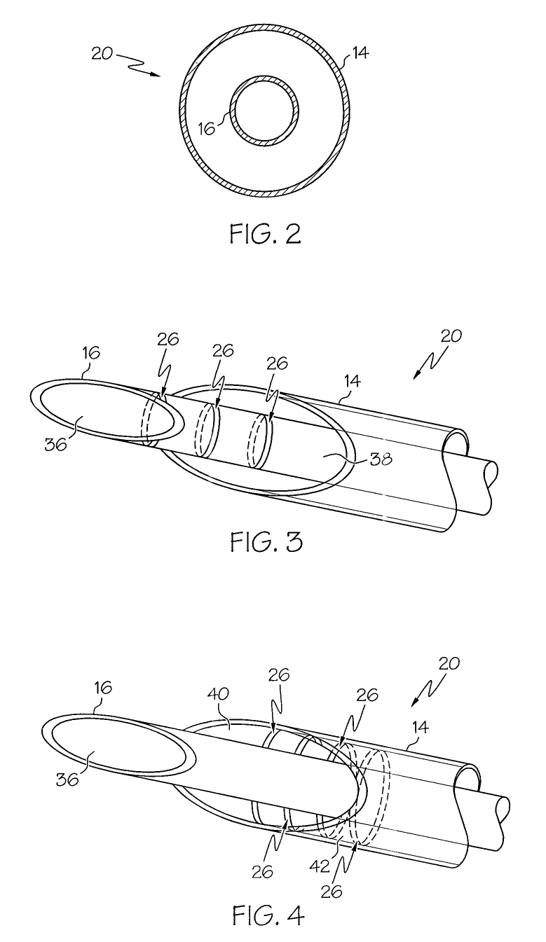

FIG. 1 is a side view illustrating a balloon catheter assembly 10, which includes an elongate and flexible catheter 20 having a proximal region 12 and a distal region 13. An exemplary catheter 20 is an over-the-wire (OTW) catheter that includes an inner tubular lumen 16 nested inside an outer tubular lumen 14. The outer lumen 16 is an inflation lumen that is adapted to facilitate transfer of an inflation fluid. The inner lumen 16 is a guidewire lumen, and is adapted to receive an elongated flexible guidewire 18 in a sliding fashion, enabling the guidewire 18 and the catheter 20 to be independently advanced or withdrawn. FIG. 2 is a cross sectional view illustrating the catheter 20, and depicts the outer inflation lumen 14 surrounding the inner guidewire lumen 16.

Returning to FIG. 1, a hub 30 is coupled to the catheter 20 at the catheter's proximal end 15, an inflatable balloon 24 is coupled to the catheter 20 at the catheter's distal end 23. The catheter's outer inflation lumen 16 is connected to and in fluid communication with both the balloon 24 and the hub 30 for the purpose of selectively inflating and deflating the balloon 24. The hub 30 includes an inflation port 17 and a guidewire port 19. The guidewire port 19 receives and feeds the guidewire 18 into the inner lumen 16. A coupling such as a luer-lock fitting or hemostatic valve 21 facilitates guidewire traversal within the guidewire lumen 26 while preventing the loss of blood or other fluids through the guidewire lumen and guidewire port. The inflation port 17 functions as a conduit for a pressurized fluid to enter the outer inflation lumen 14, which in turn facilitates transfer of the fluid to the distal end for selectively inflating and deflating the balloon 12.

In addition to the balloon 12, the catheter distal region 13 includes a distal tip 20 and marker bands 22. The balloon 24 is depicted in an expanded form in order to clearly show these components. However, before and during advancement of a catheter to a blood vessel lesion, the balloon is folded around the catheter distal end 13 and has a relatively low profile. The tip 20 is affixed as a seal at the catheter distal end 13. The tip 20 is a flexible member with a rounded nose, and is thereby adapted to guide the catheter through the tortuous pathway of a patient's vasculature while preventing damage to blood vessel walls. The marker bands 22 are located on the inner lumen 16 in the vicinity of the balloon 24. The markers 22 include a radiopaque material that can be seen using imaging techniques such as x-ray or fluoroscopy to enable their visualization during their use in the body of a patient.

Exit markers 26 are located in the catheter proximal region 12 at exact distances from the catheter distal end 23. The exit markers are used as a catheter positioning tool. The exit markers 26 are in plain view, meaning that they are formed from a material that reflects light in the visible range and can be readily seen by the naked eye of the physician before they are advanced into the catheter point of entry during catheter advancement and/or retraction through a patient's vasculature. The markers indicate how much of the catheter length has been inserted into the patient. For example, the markers allow the physician to see exactly how far it is from the catheter's point of entry to the lesion. This is particularly useful, for example, if a catheter exchange becomes necessary.

As previously discussed, conventional exit markers are printed or otherwise located on the outer shaft and consequently must be made from a material that is approved by a regulatory agency as suitable for blood contact. According to various embodiments of the invention, the exit markers 26 are located at positions along the catheter proximal region 12 that do not come into contact with blood. FIG. 3 is a cutaway perspective view illustrating a first exemplary OTW catheter 20 with exit markers 26 located on the inner lumen 16. More particularly, the inner lumen 16 has an interior wall 36 and an exterior wall 38, and the exit markers 26 are located on the inner lumen exterior wall 38. According to one exemplary embodiment, the exit markers 26 are printed on the exterior wall 38 with a durable coloring dye such as a paint or ink. The markers 26 may also be formed by spraying a dye from a spraying nozzle onto the exterior wall 38. In yet another exemplary embodiment, the exit markers 26 are metal or polymer rings that are embedded into the exterior wall 38 using, for example, a crimping technique.

As illustrated in FIG. 3, the outer tubular lumen 14 is transparent. Thus, the exit markers 26 are clearly visible to a physician while advancing and/or retracting the catheter 20 during surgery, and are as effective as markers that are conventionally formed on a catheter outer lumen. Furthermore, the inner lumen exterior wall 38 does not come into contact with blood at any time. For this reason, optimal marker materials may be selected and improved upon without concern for their biocompatibility.

Turning now to FIG. 4, a cutaway perspective view illustrates the OTW catheter 20, which according to this embodiment includes exit markers 26 located on a non-blood-contacting portion of the outer tubular lumen 14. More particularly, the outer lumen 14 includes an interior wall 40 and an exterior wall 42, and the exit markers 26 are located on the outer lumen interior wall 40. As with the previous embodiment, the outer lumen 14 is made from a transparent material, which enables a physician to plainly view the exit markers 26 while advancing and/or retracting the catheter 20 during surgery even though they are not formed on the exterior surface 42. Further, the outer lumen interior wall 40 does not come into contact with blood at any time. According to one exemplary embodiment, the exit markers 26 are printed or sprayed from a spraying nozzle onto the interior wall 40 with a durable coloring dye such as a paint or ink. Metal or polymer rings may also be crimped or otherwise embedded into the interior wall 40 to form the exit markers 26.

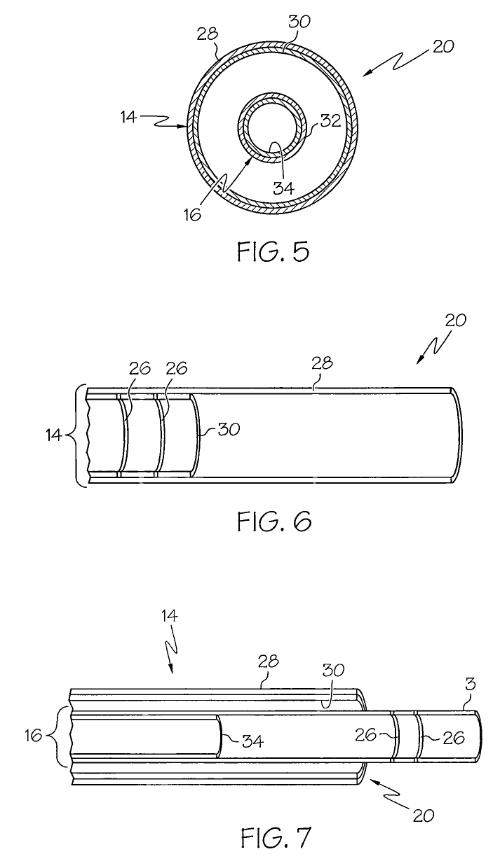

According to another exemplary embodiment, the catheter inner tubular lumen 16 and/or the catheter outer tubular lumen 14 have dual-layered architectures. FIG. 5 is a cross-sectional view illustrating the catheter inner lumen 16 that includes an outer layer 32 and an inner layer 34, and the outer lumen 14 that includes an outer layer 28 and an inner layer 30. As will be evident from the following description of the catheter 20, either the inner lumen 16 or the outer lumen 14 may have a single-layered architecture since exit markers will be located only on a lumen having a dual-layered architecture. Further, the embodiment is not limited to dual-layered lumens, and each of the outer lumen 14 and the inner lumen 16 may be formed from more than two layers of material.

Turning now to FIG. 6, a cross-sectional longitudinal view illustrating the dual-layered outer tubular lumen 14 for the OTW catheter 20 depicted in FIG. 5. As previously discussed, the outer lumen 14 includes an outer layer 28 and an inner layer 30. The outer layer 28, and preferably the inner layer 30 as well, is substantially transparent. The exit markers 26 are included with the inner layer 30 according to this exemplary embodiment. The inner layer 30 is a non-blood-contacting layer. For this reason, optimal marker materials may be selected and improved upon without concern for their biocompatibility.

With the exit marker-bearing lumen having a dual-layered architecture, the markers may be formed from a wide variety of methods. As with the previous embodiments, the exit markers 26 may be printed or sprayed from a spraying nozzle onto the inner layer 30 with a durable coloring dye such as a paint or ink, or may be formed from metal or polymer rings that are crimped or otherwise embedded into the inner layer 30. Another marker forming method is an alternating polymer extrusion process by which the exit markers 26 are integrally formed into a polymer forming the inner layer. One exemplary extrusion method includes using an extruder to form the inner layer 30 by alternating between extruding a first polymer that forms almost the entire inner layer 30, and extruding a second exit marker polymer having a different color than the first polymer. Another exemplary extrusion method includes using an extruder to form the inner layer 30 by extruding the same polymer to form the entire inner layer 30, but injecting a dye into the polymer during extrusion of the exit marker portion of the layer 30.

According to another exemplary embodiment, the exit markers are part of the dual-layered inner tubular lumen 16 depicted in FIG. 5. FIG. 7 is a cross-sectional longitudinal view illustrating both the dual-layered outer tubular lumen 14 and the dual-layered inner tubular lumen 16 for the OTW catheter 20. As previously discussed, the inner lumen 16 includes an outer layer 32 and an inner layer 34. The exit markers 26 are included with the outer layer 32, because the outer layer 32 is a non-blood-contacting layer. Any of the methods discussed above with reference to the embodiment illustrated in FIG. 6 may be used to form the exit markers 26.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

What is claimed is:

1. A catheter having non-blood-contacting exit markers, comprising:
an elongate flexible inner tubular lumen having proximal and distal regions, an outer surface, and an inner surface, the inner surface defining a guidewire passageway;
an elongate, flexible, and substantially transparent outer tubular lumen having an outer surface, and an inner surface surrounding the inner tubular lumen; and
a plurality of exit markers formed on the inner tubular lumen outer surface in the proximal region, the exit markers being visible to the naked eye through the substantially transparent outer tubular lumen.

2. The catheter according to claim 1, wherein the exit markers are printings on the inner tubular lumen outer surface.

3. The catheter according to claim 1, wherein the exit markers are ring-shaped objects.

4. The catheter according to claim 1, wherein the ring-shaped objects are embedded into the inner tubular lumen outer surface.

5. The catheter according to claim 3, wherein the ring-shaped objects are formed from a material selected from the group consisting of metals and polymers.

6. The catheter according to claim 1, wherein the inner tubular lumen comprises at least an inner layer and an outer layer, the outer layer comprising the outer surface having the plurality of exit markers formed thereon.

7. A catheter having non-blood-contacting exit markers, comprising:
an elongate flexible inner tubular lumen having outer and inner surfaces, the inner surface defining a guidewire passageway;
an elongate flexible outer tubular lumen having proximal and distal regions, a substantially transparent outer surface, and an inner surface surrounding the inner tubular lumen; and
a plurality of exit markers formed on the outer tubular lumen inner surface in the proximal region, the exit markers being visible to the naked eye through the substantially transparent outer tubular lumen outer surface.

8. The catheter according to claim 7, wherein the exit markers are printings on the outer tubular lumen inner surface.

9. The catheter according to claim 7, wherein the exit markers are ring-shaped objects.

10. The catheter according to claim 7, wherein the ring-shaped objects are embedded into the outer tubular lumen inner surface.

11. The catheter according to claim 10, wherein the ring-shaped objects are formed from a material selected from the group consisting of metals and polymers.

12. The catheter according to claim 1, wherein the outer tubular lumen comprises at least an inner layer and an outer layer, the inner layer comprising the inner surface having the plurality of exit markers formed thereon.

13. A catheter having non-blood-contacting exit markers, comprising:
an elongate flexible inner tubular lumen formed from a first polymer, the inner tubular lumen having proximal and distal regions, and outer and inner surfaces, the inner surface defining a guidewire passageway;
an elongate flexible outer tubular lumen formed from a second polymer, the outer tubular lumen having proximal and distal regions, a substantially transparent outer surface, and an inner surface surrounding the inner tubular lumen; and
a plurality of exit markers integrally formed from a third polymer, the third polymer forming a portion of the proximal region of the outer tubular lumen or the inner tubular lumen, the exit markers being visible to the naked eye.

14. The catheter according to claim 13, wherein the exit markers are a dye-injected colored polymer forming a portion of the proximal region of the outer tubular lumen or the inner tubular lumen.

15. The catheter according to claim 13, wherein the exit markers are a colored polymer extruded to form a portion of the proximal region of the outer tubular lumen or the inner tubular lumen.

16. The catheter according to claim 15, wherein the exit markers form a portion of the proximal region of the outer tubular lumen, and the third polymer forming the exit markers is different than the second polymer.

17. The catheter according to claim 15, wherein the exit markers form a portion of the proximal region of the inner tubular lumen, and the third polymer forming the exit markers is different than the first polymer.

18. The catheter according to claim 13, wherein the inner tubular lumen comprises an inner layer forming the inner surface, and an outer layer forming the outer surface and having the exit markers integrally formed therein.

19. The catheter according to claim 13, wherein the outer tubular lumen comprises an outer layer forming the outer surface, and an inner layer forming the inner surface and having the exit markers formed therein.
